(19) United States
(12) Patent Application Publication
(10) Pub. No.: US 2024/0016604 A1
(43) Pub. Date: Jan. 18, 2024

(54) CATHETER SYSTEMS WITH HYDRAULIC SHOCK ARRESTOR

(71) Applicant: EDWARDS LIFESCIENCES CORPORATION, Irvine, CA (US)

(72) Inventor: Michael C. Murad, Lake Mathews, CA (US)

(21) Appl. No.: 18/476,940

(22) Filed: Sep. 28, 2023

Related U.S. Application Data
(63) Continuation of application No. PCT/US22/23972, filed on Apr. 8, 2022.
(60) Provisional application No. 63/173,167, filed on Apr. 9, 2021.

(51) Int. Cl. A61F 2/24

(52) U.S. Cl. CPC A61F 2/2433; A61F 2250/0073

(57) ABSTRACT

Catheter systems and methods including a hydraulic shock arrestor. A system may include an inflatable balloon for insertion within a patient's body. The inflatable balloon may be configured to be inflated with a fluid within the patient's body. The system may further include an elongate shaft. The elongate shaft may be configured to extend within the patient's body. The system may further include a fluid conduit. The system may further include a hydraulic shock arrestor configured to mitigate pressure surge within the fluid conduit.

### CROSS-REFERENCE TO RELATED APPLICATIONS

This application is a continuation of International Application No. PCT/US2022/023972, filed Apr. 8, 2022, which designates the United States and was published in English by the International Bureau on Oct. 13, 2022 as WO2022/217025, which claims the benefit of U.S. Provisional Application No. 63/173,167, filed Apr. 9, 2021, the entire disclosure of each of which being incorporated herein by reference for all purposes.

### FIELD

The present disclosure relates to medical catheters and more particularly, to catheter systems having a hydraulic shock arrestor.

### DESCRIPTION OF THE RELATED ART

Catheters are used in a variety of interventional procedures for delivering therapeutic means to an area of a human body being treated (e.g., an organ, a blood vessel). In certain procedures, the catheters may have balloons that may be guided into the treatment site and inflated once at the treatment site to expand a blocked blood vessel, place treatment means (e.g., a heart valve, a stent) and/or deliver surgical tools to the treatment site. Additionally, balloon catheters may also used to retrieve treatment means and/or surgical tools from passageways of the body.

Generally, a fluid actuator capable of pumping fluid may be used to inflate a balloon of a catheter. The fluid may be pushed out via a plunger inside a barrel of the fluid actuator. When the plunger bottoms out inside the barrel, all fluid inside the barrel is pushed out and the plunger ceases movement. The cessation of movement of the fluid by the fluid actuator may cause a pressure surge or hydraulic shock that may be known as a water hammer effect. The amplitude of the pressure surge wave may be directly proportional to the inflation rate of the balloon. As such, faster inflation may lead to a larger surge wave. The pressure surge may have various negative results, which may include damage to sensitive components (e.g., a pressure sensor) or fragile connections along a fluid conduit of the catheter. Further, a pressure surge may result in the balloon bursting, implant failure, or increased risk of annular rupture. These negative results may jeopardize a patient's health.

The present systems and methods may relate to catheter systems that may include a hydraulic shock arrestor. The systems and methods may include an inflatable balloon that may be inserted within a patient's body and inflated with fluid within a patient's body. A fluid conduit may extend between the inflatable balloon and a fluid actuator that may be configured to convey movement of the fluid to the inflatable balloon from the fluid actuator to inflate the inflatable balloon. A hydraulic shock arrestor may mitigate pressure surge within the fluid conduit. By mitigating pressure surge, the hydraulic shock arrestor may prevent or reduce risk of damage to sensitive components or fragile connections along a fluid conduit of the catheter, or may prevent or reduce risk of the inflatable balloon bursting, or implant failure, or may reduce risk of annular rupture.

One or more examples of the present disclosure may include a catheter system. The system may include an inflatable balloon for insertion within a patient's body. The inflatable balloon may be configured to be inflated with a fluid within the patient's body. The system may further include an elongate shaft. The elongate shaft may be configured to extend within the patient's body. The elongate shaft may have a distal end portion configured to couple to the inflatable balloon and a proximal end portion. The system may further include a fluid conduit. The fluid conduit may be configured to extend between the inflatable balloon and a fluid actuator. The fluid conduit may be further configured to convey movement of the fluid to the inflatable balloon from the fluid actuator to inflate the inflatable balloon. The system may further include a hydraulic shock arrestor configured to mitigate pressure surge within the fluid conduit.

One or more examples of the present disclosure may include a method. The method may include extending an elongate shaft of a catheter system within a portion of a patient's body. The catheter system may include an inflatable balloon that is coupled to a distal end portion of the elongate shaft. The inflatable balloon may be configured to be inflated with a fluid. The catheter system may include a fluid actuator configured to move the fluid to inflate the inflatable balloon. The catheter system may include a fluid conduit extending between the inflatable balloon and the fluid actuator. The fluid conduit may convey movement of the fluid to the inflatable balloon from the fluid actuator. The catheter system may include a hydraulic shock arrestor configured to mitigate pressure surge within the fluid conduit. The method may further include positioning the inflatable balloon at an inflation site within the patient's body. The method may further include inflating the inflatable balloon utilizing the fluid actuator.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar examples.

FIG. 1 illustrates a side schematic view of a catheter system according to an example of the present disclosure.

FIG. 2 illustrates a schematic view of a fluid conduit according to an example of the present disclosure.

FIG. 3 illustrates a cross-section view of the hydraulic shock arrestor and the fluid actuator of FIG. 1 when a plunger of the fluid actuator is bottomed out according to an example of the present disclosure.

FIG. 4A illustrates a close-up side view of a connector of a catheter system having an integrated hydraulic shock arrestor according to an example of the present disclosure.

FIG. 4B illustrates a cross-section view of the connector of FIG. 4A according to an example of the present disclosure.

FIG. 5A illustrates a close-up side view of a fluid actuator of a catheter system having an integrated hydraulic shock arrestor according to an example of the present disclosure.

FIG. 5B illustrates a cross-section view of the fluid actuator of FIG. 5A according to an example of the present disclosure.

FIG. 6 illustrates an isolated cross-section view of a plunger of a fluid actuator of a catheter system according to an example of the present disclosure.

FIG. 7 illustrates a side schematic view of a balloon system having a hydraulic shock arrestor including an expandable section along an elongate shaft according to an example of the present disclosure.

FIG. 8A illustrates a close-up cross section view of a hydraulic shock arrestor shown in FIG. 7.

FIG. 8B illustrates a close-up cross section view of the hydraulic shock arrestor shown in FIG. 7 deflected from the position shown in FIG. 8A.

FIG. 9 illustrates a side view of a prosthetic valve according to an example of the present disclosure.

FIG. 10 illustrates a top view of the prosthetic valve shown in FIG. 9.

FIG. 11 illustrates a top view of the prosthetic valve shown in FIG. 9 with prosthetic leaflets moved from the position shown in FIG. 10.

FIG. 12 illustrates a side view of a catheter system in the form of a delivery apparatus.

FIG. 13 illustrates a schematic view of an implant advanced to an implantation site.

FIG. 14 illustrates a schematic view of an implant deployed to an implantation site.

The catheter systems described herein may include a hydraulic shock arrestor to mitigate pressure surge within a fluid conduit of the system. In examples, the hydraulic shock arrestor may be a water hammer arrestor. During a medical procedure (e.g., deployment of a prosthetic heart valve, angioplasty), a catheter may be inserted into a patient's body where treatment is desired, such as a passageway (e.g., a blood vessel) of the patient. An inflatable balloon may be inflated within the patient's passageway. A fluid actuator may be used to inflate the inflatable balloon with fluid. The fluid may be liquid. A plunger of the fluid actuator may push out the fluid inside a barrel of the fluid actuator. When no fluid is left within the fluid actuator and the plunger bottoms out, the movement of the fluid may abruptly cease. The cessation of the movement may result in a hydraulic shock. The hydraulic shock arrestor may advantageously mitigate pressure surge. In examples, the hydraulic shock arrestor may prevent or reduce risk of damage to sensitive components or fragile connections along a fluid conduit of the catheter system as well as medical complications. Further, the hydraulic shock arrestor may advantageously permit inflating the balloon at a desired rate with reduced risk of causing a pressure surge.

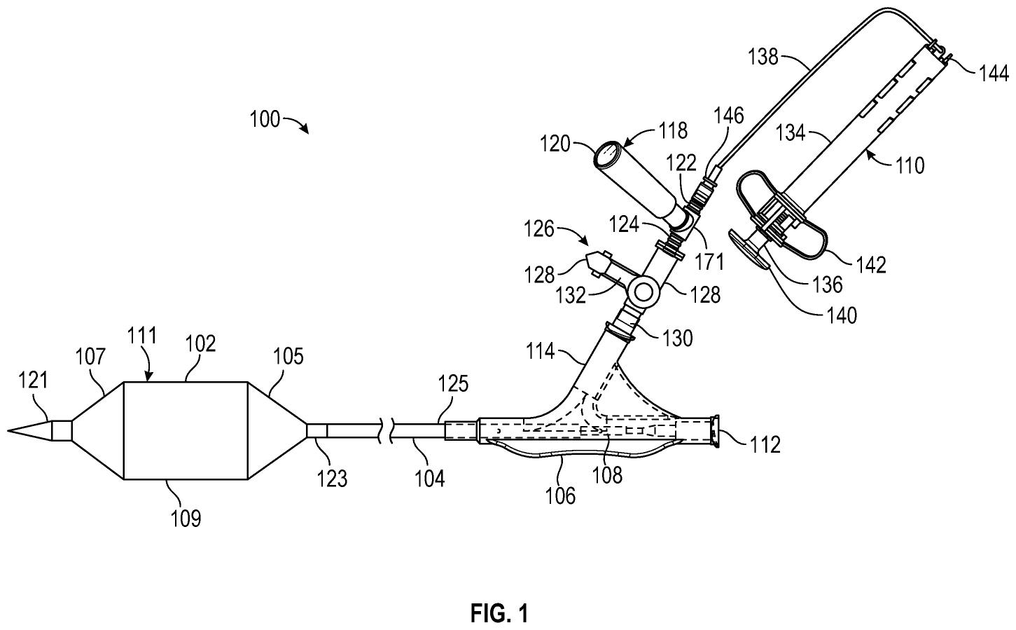

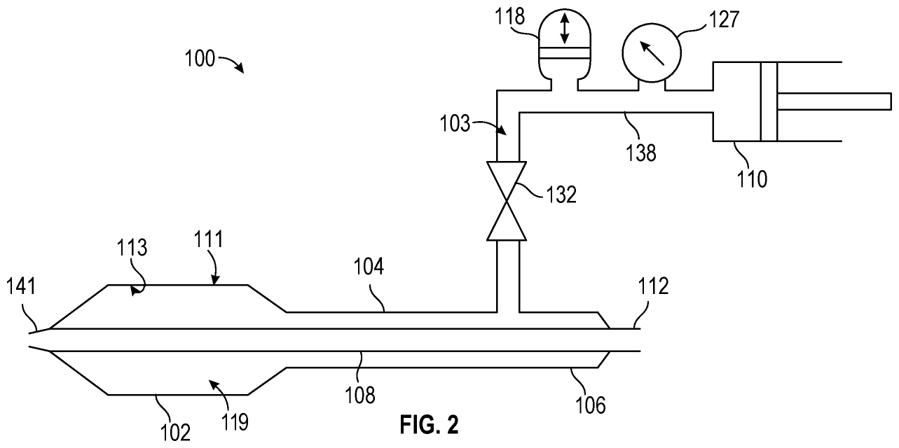

FIG. 1 illustrates a side view of a catheter system 100 according to an example of the present disclosure. The catheter system 100 may include an inflatable balloon 102 and an elongate shaft 104. The catheter system 100 may further include a connector 106 that may be positioned at a proximal end portion of the elongate shaft 104. The catheter system 100 may further include a fluid conduit 103 (marked in FIG. 2) and a hydraulic shock arrestor 118 that may be configured to mitigate pressure surge within the fluid conduit 103. In examples, the catheter system 100 may further include a fluid actuator 110 that may be configured to move the fluid to inflate the inflatable balloon 102.

The inflatable balloon 102 may be configured for insertion with a patient's body and may be configured to be inflated with a fluid within a patient's body. The inflatable balloon 102 may include a proximal portion 105, a distal portion 107, and a central portion 109 positioned between the proximal portion 105 and the distal portion 107.

In examples, the central portion 109 of the inflatable balloon 102 may have a cylindrical shape as shown in FIG. 1. The proximal portion 105 may have a shape that tapers radially outward in a distal direction to the central portion 109. The distal portion 107 may have a shape that tapers radially inward in a distal direction from the central portion 109 to a distal tip of the inflatable balloon 102. In examples, other configurations of inflatable balloons may be utilized, including tapered shapes or dumbbell shapes, or other shapes based on the desired use of the inflatable balloon. The central portion 109 in examples may comprise a pressing portion configured to apply a force to a surface external of the inflatable balloon 102.

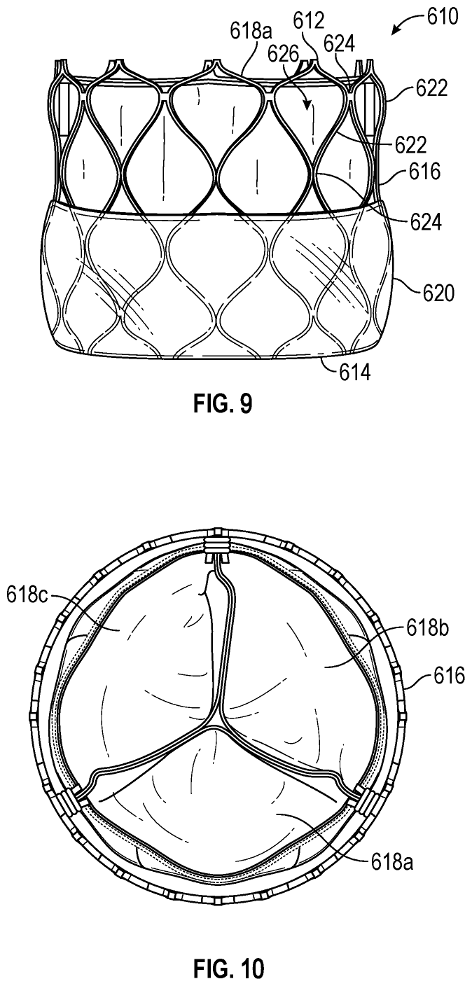

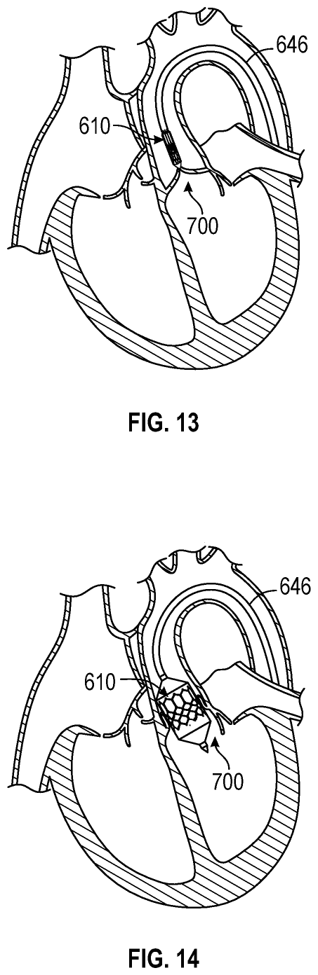

The inflatable balloon 102 may include an outer surface 111 and an inner surface 113 (marked in FIG. 2) that faces opposite the outer surface 111. The outer surface 111 may be configured to apply a force to a surface external to the inflatable balloon 102 to dilate the surface or otherwise perform an operation upon the surface. For example, the outer surface 111 at the central portion 109 may be configured for an implant to be positioned upon for delivery to an implantation site within a patient's body. The implant, for example, may be crimped upon the inflatable balloon 102 with the inflatable balloon 102 in an unexpanded or uninflated state and then the inflatable balloon may be inflated with a fluid to expand the implant. FIGS. 9-11 for example, illustrate an exemplary implant that may be dilated and expanded according to examples herein, and FIGS. 13-14 illustrate an exemplary delivery procedure.

The inner surface 113 may face a fluid chamber 119 (marked in FIG. 2) that may be configured to be filled with fluid to inflate the inflatable balloon 102. A wall of the inflatable balloon 102 may surround the fluid chamber 119. The fluid chamber 119 may form a portion of the fluid conduit 103 that is utilized to inflate the balloon 102.

The inflatable balloon 102 may be configured to be inflated with a fluid to dilate a surface within the patient's body. For example, the inflatable balloon 102 may be in an uninflated state (for example as shown with the balloon 658 in FIG. 12) and then advanced towards and positioned at an inflation site within the patient's body. The inflatable balloon 102 may then be inflated to dilate a surface, which may be a constricted artery, leaflets of a native valve (such as a native heart valve) within the patient's body, or another surface. In examples, the surface may be an inner surface of an implant, such as a prosthetic heart valve or other form of implant (e.g., a stent or other implant). The inflatable balloon 102 may then be deflated and may be withdrawn from the inflation site and from the patient's body.

The inflatable balloon 102 may have a variety of compositions. The inflatable balloon 102 may comprise a non-compliant or semi-compliant balloon in examples and may comprise a compliant balloon in examples. The inflatable balloon 102 may be made to exert high-pressure, mid-pressure, or low-pressure in examples. The inflatable balloon 102 in examples may be elastomeric. High-pressure balloons may be used to open a blockage or dilate the vasculature and in examples may be made from polyester, nylon, and/or other forms of material. Mid-pressure balloons may be more compliant and flexible compared to high-pressure balloons to ease delivery. Mid-pressure balloons may be made from Pebax, higher-durometer polyurethanes, and/or other forms of material. Elastomeric balloons in examples may fully conform to the shape of its environment and stretch 100% to 800%. Elastomeric balloons may be made from polyurethane, silicone, and/or other forms of material. Various other compositions of balloons may be utilized as desired.

The distal portion 107 of the inflatable balloon 102 may couple to a nose cone 121 that may comprise a leading tip of the catheter system. The proximal portion 105 of the inflatable balloon 102 may couple to a distal end portion 123 of the elongate shaft 104.

The elongate shaft 104 may be configured to extend within the patient's body and may have a distal end portion 123 configured to couple to the inflatable balloon 102 and may include a proximal end portion 125. The elongate shaft 104 may have a length from a distal end of the elongate shaft to the proximal end. The elongate shaft 104 may have a cylindrical shape or another shape as desired. The elongate shaft 104 may be configured to be rigid or flexible to allow for the elongate shaft 104 and the inflatable balloon 102 to advance to a desired treatment site within the patient's body. For example, the elongate shaft 104 may be configured to advance through the patient's vasculature, including the patient's arteries, to be delivered to the desired treatment site.

The elongate shaft 104 may have sufficient length to position the inflatable balloon 102 at the desired treatment site, yet with the proximal end portion 125 remaining exterior to the patient's body for use and manipulation by a user, such as a surgeon or other medical technician.

The proximal end portion 125 of the elongate shaft 104 may couple to a connector 106, which may be external to the patient's body during treatment, and may comprise a handle that may be gripped by a user during a treatment procedure.

In examples, the elongate shaft 104 may be made from a variety of materials. Such materials may include a polymer material such as, as non-limiting examples, silicone rubber, latex, polyurethane (PUR), polyethylene terephthalate (PET), fluorinated ethylene propylene (FET), or silicone, or other forms of materials. In some examples, the elongate shaft 104 may be made from polytetrafluoroethylene (PTFE, or Teflon). In other examples, the elongate shaft 104 may be made from a thermoplastic elastomer, such as thermoplastic urethanes and polyether block amides (PEBA). Other materials may be utilized as desired.

The elongate shaft 104 may retain components that may be utilized to inflate the inflatable balloon 102 and may be utilized to perform other operations of the catheter system 100 including deflection of the elongate shaft 104. For example, referring to FIG. 12, a control mechanism may extend along the elongate shaft 104 and may be utilized to deflect the elongate shaft 104 to the desired orientation within the patient's body for treatment. Other components may extend along the elongate shaft 104 as desired.

At least a portion of the fluid conduit 103 may extend along the elongate shaft 104. FIG. 2, for example, illustrates a schematic view of a configuration of a fluid conduit 103 that may be utilized according to examples herein. Referring to FIG. 2, the fluid conduit 103 may be configured to extend between the inflatable balloon 102 and a fluid actuator 110, and configured to convey movement of the fluid to the inflatable balloon 102 from the fluid actuator 110 to inflate the inflatable balloon 102. In examples, the fluid conduit 103 may extend from the fluid actuator 110, and along components such as the hydraulic shock arrestor 118 and other components such as a sensor 127 (not visible in FIG. 1) or other components that may be positioned along the fluid conduit 103. The fluid conduit 103 may pass through components such as the valve or valve switch 132. The fluid conduit 103 may pass through the connector 106 and along the elongate shaft 104 to reach the inflatable balloon 102. At least a portion of the fluid conduit 103 may extend through the elongate shaft 104. In examples, the fluid chamber 119 may comprise a portion of the fluid conduit 103. The configuration of the fluid conduit 103 shown in FIG. 2 is exemplary in nature and other configurations of fluid conduits may be utilized as desired. For example, the fluid conduit may include multiple branches or connections that may extend to various other components or termination points of the fluid conduit. A variety of configurations of fluid conduits may be utilized as desired.

The fluid conduit 103 may comprise tubing or portions of components that include the conduit, or may comprise portions of components of the catheter system 100 that include a conduit. The fluid conduit 103 may be configured for inflation of the inflatable balloon 102 or may be configured for deflation of the inflatable balloon 102 or a combination of inflation and deflation. The fluid conduit 103 may be utilized for other purposes as desired, including but not limited to transfer of fluid to sensors or other devices along the fluid conduit 103.

Referring back to FIG. 1, the fluid actuator 110 may be configured to move the fluid to inflate the inflatable balloon. For example, the fluid actuator 110 may be a syringe as shown in FIG. 1. Other examples of the fluid actuator 110 may be an injector or a pump, or another form of a device for inflating the inflatable balloon 102. The fluid actuator 110 may be actuated manually or automatically. For example, an auto-syringe or an automatic pump may be utilized to move the fluid to inflate the inflatable balloon in examples, among other devices.

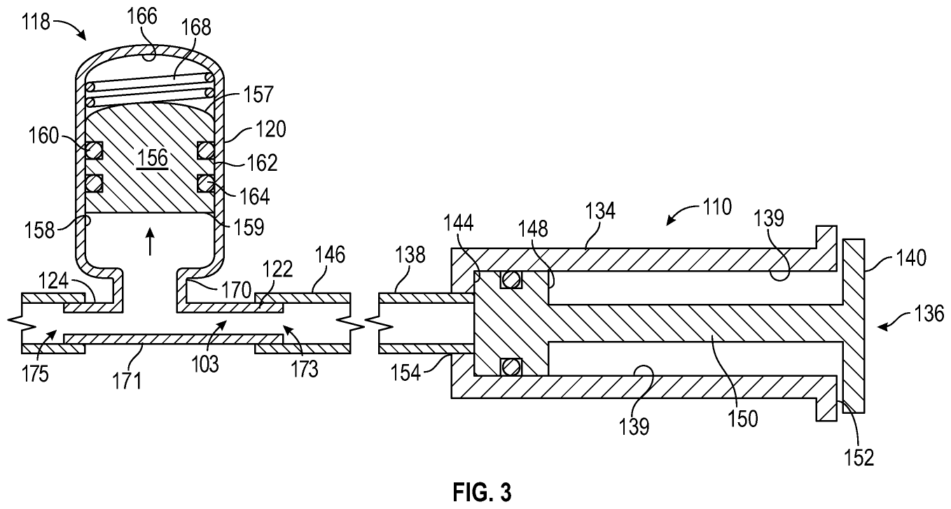

As shown in FIG. 1, the fluid actuator 110 may include a barrel 134 and a plunger 136. The barrel 134 may contain fluid to be delivered to the inflatable balloon 102. The fluid within the barrel 134 may be moved towards the inflatable balloon 102 to pass other fluid within the fluid conduit 103 (e.g., within the tubing or elongate shaft 104) into the inflatable balloon 102. The fluid conduit 103 may convey such movement of the fluid to the inflatable balloon 102 from the fluid actuator 110 to inflate the inflatable balloon 102. The fluid may be expelled from the barrel 134 by lowering the plunger 136 through the barrel 134 and squeezing out the fluid. The plunger 136 may be slidably engaged with the barrel 134 of the fluid actuator 110 and may slide up and down the barrel 134 while maintaining a seal with inner walls 139 of the barrel 134 (marked in FIG. 3). The plunger 136 may be positioned fully or partially within the barrel 134.

A handle 140 or pressing surface of the plunger 136 may extend out of the barrel 134. The handle 140 may be pressed to push and pull the plunger 136. The fluid actuator 110 in examples may have wings 142 to hold the fluid actuator 110 with reinforced grip.

FIG. 3 illustrates a cross-section view of the fluid actuator 110. The plunger 136 may have a base 148, a shaft 150, and the handle 140. The handle 140 may be connected to the base 148 via the shaft 150. The handle 140 may extend out of the barrel 134 through a proximal end 152 of the fluid actuator 110. The base 148 may form a seal with the inner walls 139 of the barrel 134. The seal may prevent the fluid from escaping towards the proximal end 152. The sealing material may be rubber, polytetrafluoroethylene, polyethylene, and/or another form of material. The seal may be liquid proof and/or gas proof. When the base 148 is bottomed out following expulsion of the fluid out of the barrel 134, the base 148 may seal a barrel outlet 154. The barrel outlet 154 may be an opening at the bottom end 144. The barrel outlet 154 may connect the barrel 134 to a fluid tube 138.

Referring back to FIG. 1, the fluid actuator 110 may be configured to inflate the inflatable balloon 102 and may be configured to deflate the inflatable balloon 102, for example, by withdrawing the plunger 136 from the barrel 134.

In examples in which the fluid actuator 110 comprises a pump or another form of fluid actuator, the configuration of the fluid actuator 110 may vary from the configuration shown in FIG. 1.

The fluid actuator 110 may be coupled to a tube 138 that may surround the fluid conduit 103 (see FIG. 2 for example). The tube 138 may be connected to the barrel 134. The fluid tube 138 may extend from a bottom end 144 of the barrel 134. Once the fluid exits the barrel 134, it may travel through the fluid tube 138. In examples, the fluid actuator 110 may be positioned at a proximal end portion of the fluid conduit 103. The tube 138 may extend from the fluid actuator 110 to the hydraulic shock arrestor 118.

Various connectors or other components may be utilized to link the fluid actuator 110 to the inflatable balloon 102 for fluid transfer. FIG. 1, for example, illustrates a connector 126 in the form of a luer connector 126 that may be configured to couple the fluid actuator 110 to the connector 106, which in turn couples the fluid actuator 110 to the elongate shaft 104 and the inflatable balloon 102. Other connectors or components may be utilized as desired.

The luer connector 126 may have a valve switch 132 or a stopcock that permits flow from a selected inlet 128 at a time or cuts flow from all inlets 128. The luer connector 126 may have one outlet 130 or multiple outlets as desired. The outlet 130 may be inserted into a balloon inflation port 114 of the connector 106 as desired. The luer connector 126 may be made from plastic materials, such as by plastic injection, or another material as desired. The luer connector 126 may be a releasable connector in examples. The fluid conduit 103 may extend through the luer connector 126.

The connector 106 may connect to the proximal portion of the elongate shaft 104. The connector 106 in examples may be a Y-connector (i.e., have a connection configured as a “Y”). The connector 106 may have one or more inlets and one outlet. The connector 106 may be a releasable connector and may be a luer connector in examples. The fluid conduit 103 may extend through the connector 106.

In examples, the connector 106 may include a balloon inflation port 114 for receiving fluid from the fluid actuator 110. The connector 106 may further include a guide wire lumen 108 that may pass through the connector 106 and may pass through the elongate shaft 104 (as represented in FIG. 2). The guide wire lumen 108 may extend along the elongate shaft 104 and may be configured to receive a guide wire. The guide wire lumen 108 may have a distal end including an opening 141 (marked in FIG. 2) and a proximal end including a guide wire lumen port 112.

In examples, the fluid passing through the balloon inflation port 114 may be in a separate lumen (comprising the fluid conduit 103) from the guide wire lumen 108. Further, the guide wire lumen 108 may be in a separate lumen from other lumens of the elongate shaft 104, which may comprise a lumen including one or more pull wires of a control mechanism among other components. In examples, combinations of lumens may extend along the elongate shaft 104.

The catheter system 100 may include a hydraulic shock arrestor 118 that may be configured to mitigate pressure surge within the fluid conduit 103. The hydraulic shock arrestor 118 may have a variety of forms, including expandable portions that may be configured to receive the pressure surge and expand to mitigate the pressure surge. For example, pistons, diaphragms, or other expandable components may be configured to receive the pressure surge. The hydraulic shock arrestor 118 may comprise a water hammer arrestor.

FIG. 3 for example, illustrates an exemplary close up view of a hydraulic shock arrestor 118 that may be utilized according to examples herein. The hydraulic shock arrestor 118 may include a chamber 120 and may include a piston 156 located within the chamber 120. The piston 156 may be configured to slide within the chamber 120 relative to the inner walls 158 of the chamber 120. The piston 156 may be configured to slide up and down the chamber 120 while maintaining a seal with inner walls 158 of the chamber 120. In examples, the piston 156 may be sealingly engageable with the inner walls 158 with at least one seal ring 164 configured to create a seal between the piston 156 and the inner walls 158. The chamber 120 in examples may have a bottleneck 170 to retain the piston 156. In some examples, the chamber 120 may have a piston retainer extending into the chamber 120 from the inner walls 158.

In examples, the piston 156 may be shaped to complement the shape of the inner walls 158. The chamber 120, for example, may be cylindrical. In some examples, the chamber 120 may be a rectangular prism, a square prism, or the like. The piston 156 may have at least one groove 160 on its outer surface 162. The at least one groove 160 may be an annular groove for fitment of the seal ring 164. The seal ring 164 may be rubber, silicon, polyurethane, or another material. In some examples, the outer surface 162 may include a sealant made from a sealing material.

In examples, the chamber 120 may have a gas within. For example, the gas may be air or another gas as desired. The chamber 120 may be filled with gas to resist pressure. The piston 156 may be positioned in between and in contact with the gas and the fluid of the fluid conduit 103. For example, the piston 156 may include a first side 157 configured to be in contact with the gas and a second side 159 configured to be in contact with the fluid within the fluid conduit 103. The gas may be positioned between the first side 157 and a top end 166 of the chamber 120. The gas in examples may comprise a compressed air.

In some examples, an inner spring 168 may be located between the piston 156 and the top end 166 of the chamber 120. The inner spring 168 may be configured to absorb pressure and absorb the pressure surge in examples solely or in combination with the gas in the chamber 120.

In operation, the piston 156 may float on the fluid from the fluid conduit 103 prior to a hydraulic shock being produced. Other configurations of hydraulic shock arrestors may be utilized in examples as desired.

The hydraulic shock may be produced in a variety of manners. For example, FIG. 3 illustrates the plunger 136 of the fluid actuator 110 bottomed out according to an aspect of the present disclosure. The bottoming out of the plunger 136 may cause a sudden change in momentum of the fluid within the fluid conduit 103, which may thus result in a pressure surge. The bottoming out of the plunger 136 may occur during inflation of the inflatable balloon 102 and may comprise a sudden cessation of movement of the plunger 136 and the fluid within the fluid conduit 103. Such an effect may be known as a water hammer effect that comprises a hydraulic shock, a pressure surge or wave caused when the fluid in motion is forced to change momentum, stop or change direction, suddenly.

Hence, the hydraulic shock may cause a surge in fluid pressure within the fluid conduit 103. The pressure surge may cause a spike in peak pressure over time. The spike may be approximately 0.5 atm, or may be a greater or lesser amount based on the variation in the momentum of the fluid. In examples, the amplitude of the pressure surge may be proportional to the rate of inflation. In examples, other causes may produce the hydraulic shock, including turning off an inflation pump, ceasing movement of a plunger, or quickly closing a valve, among other causes.

The hydraulic shock arrestor 118 may absorb the pressure surge. For example, the fluid may raise the piston 156 due to the force of the hydraulic shock. The raised piston 156 may compress the air in examples. The compressed air may counter and resist the movement of the piston 156, thereby absorbing the pressure surge of the fluid. FIG. 3 shows the piston 156 raised. In examples, the inner spring 168 may be compressed to absorb the shock.

The hydraulic shock accordingly may have a reduced possibility of continuing along the fluid conduit 103 to potentially damage the inflatable balloon 102, or other components of the catheter system 100 including the elongate shaft 104 and any tubing or connectors along the fluid conduit 103.

In examples, the hydraulic shock arrestor 118 may mitigate pressure surge within the fluid conduit 103 to reduce the possibility of damage to sensors or improper readings of one or more sensors along the fluid conduit 103. For example, FIG. 2 illustrates a sensor 127 in the form of a pressure sensor that may be positioned along the fluid conduit 103 and configured to sense a pressure of the fluid within the fluid conduit 103. The hydraulic shock arrestor 118 may mitigate the pressure surge within the fluid conduit 103 such that a pressure surge has a reduced effect upon the pressure reading of the pressure sensor. Other forms of sensors may be utilized in other examples. Further, the possibility of damage to sensors may be reduced in examples. The possibility of damage to other components such as connectors may be reduced in examples. For example, a fragile connector or other component may be proximate the source of the hydraulic shock and the hydraulic shock arrestor 118 may reduce the possibility of damage to such a connector or component by mitigating the pressure surge within the fluid conduit.

The hydraulic shock arrestor 118 may be positioned in a variety of locations along the fluid conduit 103. In examples, the hydraulic shock arrestor 118 may be positioned proximate a source of the hydraulic shock, such as the fluid actuator 110 as shown in FIG. 3. Other locations may be utilized as desired. The hydraulic shock arrestor 118 for example, may be positioned between a source of hydraulic shock such as a fluid actuator 110 and a component in which the hydraulic shock would not be desired such as an inflatable balloon 102, or the elongate shaft 104, or a sensor 127, or a connector, in examples.

FIG. 3 illustrates an example in which the hydraulic shock arrestor 118 is positioned along the fluid conduit 103 at a position between the fluid actuator 110 and the elongate shaft 104. In examples, the hydraulic shock arrestor 118 may be positioned between the fluid actuator 110 or other source of hydraulic shock (such as a valve that may close) and a sensor 127, which may comprise a pressure sensor. Other locations may be utilized as desired.

In examples, the hydraulic shock arrestor 118 may be releasably coupled to portions of the fluid conduit 103. As such, the hydraulic shock arrestor 118 may be added in line with the fluid conduit 103 as a component of the fluid conduit of the catheter system 100.

FIG. 3 for example, illustrates the hydraulic shock arrestor 118 may be coupled to a tube 171. The tube 171 may extend around a portion of the fluid conduit 103 as shown in FIG. 3. The tube 171 may have an inlet connector 122 with a first opening 173 and an outlet connector 124 with a second opening 175. The outlet connector 124 is configured to couple to the elongate shaft 104 and the inlet connector 122 is configured to couple to the fluid actuator 110. The fluid conduit 103 extends from the first opening 173 to the second opening 175. The chamber 120 of the hydraulic shock arrestor 118 is configured to receive the fluid from the fluid conduit 103 through the bottleneck 170.

In examples, the chamber 120, the inlet connector 122, and the outlet connector 124 may be comprise a unitary body. The unitary body, for example, may be made from plastic or a metal such as copper, brass, and aluminum, among other materials. The unitary body may be added in line with the fluid conduit system of the catheter system 100.

The hydraulic shock arrestor 118 may be coupled in line by connecting the connectors 122, 124. For example, referring to FIG. 1, the tube 138 may have a distal fluid tube outlet 146 that couples to the inlet connector 122. For example, the fluid tube outlet 146 and the hydraulic shock arrestor inlet connector 122 may be threaded and screwed into place to establish a secure connection. The outlet connector 124 may couple to an inlet 128 of the luer connector 126 in examples, which may have an outlet 130. The outlet 130 may couple to the balloon inflation port 114 of the connector 106.

The connector 126 may be configured to couple the hydraulic shock arrestor 118 to the elongate shaft 104. The connector 126 may comprise a releasable connector, such that the hydraulic shock arrestor 118 may be added in line with the fluid conduit 103, and released from the fluid conduit 103 if desired. For example, during assembly, a connector 126 may be utilized to connect the hydraulic shock arrestor 118 in line with the remainder of the fluid conduit 103. The inlet connector 122 and outlet connector 124 may further comprise releasable connectors that allow the hydraulic shock arrestor 118 to be positioned in line.

In an exemplary operation, the elongate shaft 104 of the catheter system 100 may be extended within a portion of the patient's body. For example, to insert the inflatable balloon 102 into a treatment area of a patient, the patient may first receive a needle puncture to the skin located proximate to the treatment area. A guide wire may be inserted through and extend out of the guide wire lumen 108. The guide wire may be inserted through the guide wire lumen port 112 of the connector 106. The guide wire may be advanced to extend beyond the treatment area to ensure coverage of the entirety of the treatment area. The elongate shaft 104 may be sleeved over the guide wire and guided towards the treatment area by the guide wire. The elongate shaft 104 may be advanced until the inflatable balloon 102 in a deflated state is at the treatment site or inflation site.

The catheter system 100 may be an over-the-wire balloon catheter where the guide wire tracks along the full length of the guide wire lumen 108. Alternately, the catheter system 100 may be a rapid exchange balloon catheter where the guide wire extends along a short section of the guide wire lumen 108 to save time. Alternately, the catheter system 100 may be a fixed-wire balloon catheter that has a wire core to advance the catheter to the treatment site in lieu of a guide wire and a guide wire lumen 108. Other forms of catheter systems may be utilized in examples.

In examples, the elongate shaft 104 may be advanced by using the connector 106 as a handle. In some examples, there may be a handle external to the connector 106. The inflatable balloon 102 may positioned at the inflation site within the patient's body. The inflatable balloon 102 may be inflated utilizing the fluid actuator 110.

The inflation may be rapid, which may result in a possible hydraulic shock due to a rapid cessation of the flow of the fluid upon inflation. In the event of a hydraulic shock, the hydraulic shock arrestor 118 may be utilized to mitigate a pressure surge within the fluid conduit 103, as discussed herein. The inflatable balloon may further be deflated rapidly.

In examples, the inflatable balloon 102 may create space for treatment at the treatment site. For example, the inflatable balloon 102 may clear a blockage at the treatment site. The inflatable balloon 102 may dilate leaflets such as heart valve leaflets prior to implantation of an implant. In examples, the inflatable balloon 102 may be utilized to deploy a device such as an implant, such as a prosthetic heart valve as shown in FIGS. 13 and 14 or other form of implant.

The configuration of the hydraulic shock arrestor 118 and the catheter system 100 may be varied in examples as desired. For example, in examples the hydraulic shock arrestor 118 may be positioned in other locations than shown in FIGS. 1-3.

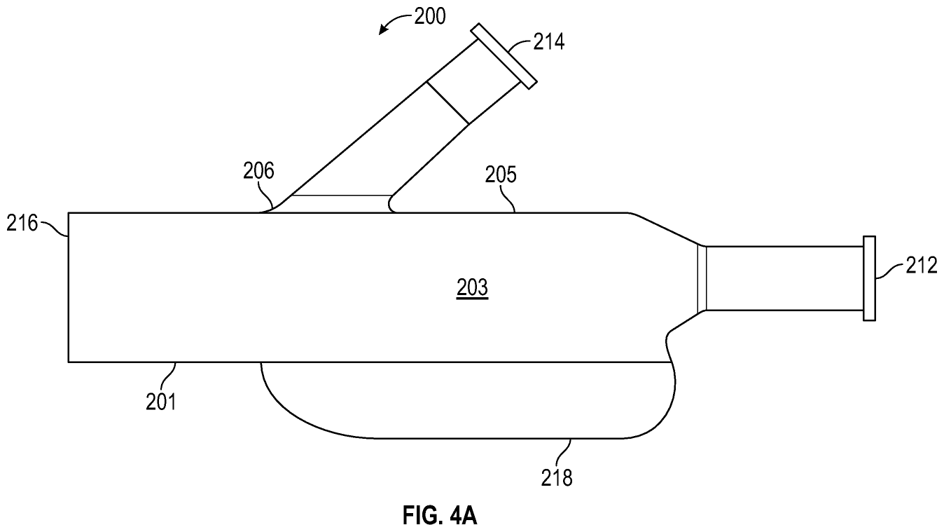

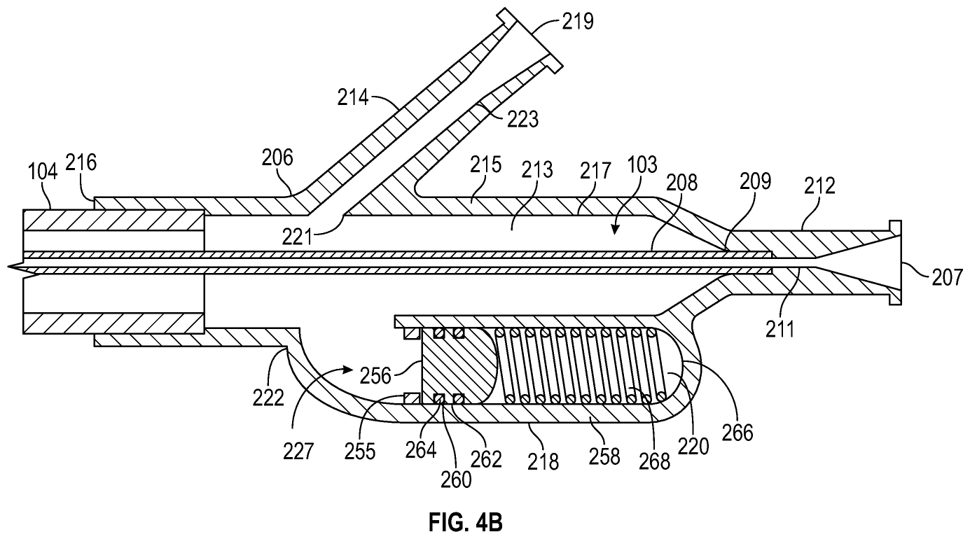

FIG. 4A, for example, illustrates a close-up side view of a connector of a catheter system 200. The connector may comprise a handle 206 that is coupled to the proximal end portion 125 of the elongate shaft 104 as shown in FIG. 1 for example. The hydraulic shock arrestor 218 may be positioned on the handle 206.

The handle 206 may include sides 203 and a top 205. The handle 206 may include a balloon inflation port 214 and a guide wire lumen port 212, similar to the connector 106 shown in FIG. 1. The hydraulic shock arrestor 218 may extend from a bottom 201 of the handle 206 as shown in FIG. 4A, although other locations (e.g., side or top) may be utilized as desired.

FIG. 4B illustrates a cross-section view of the handle 206 according to an example of the present disclosure. The balloon inflation port 214 and guide wire lumen port 212 are visible.

The handle 206 may have a handle outlet 216. The handle outlet 216 may output the inputs of the balloon inflation port 214 and the guide wire lumen port 212. The guide wire lumen port 212 may be positioned such that a guide wire may be inserted and passed straight through the handle 206 to exit out of the handle outlet 216 with no bending or minimal bending. The balloon inflation port 214 may extend from the top 205 of the handle 206. The balloon inflation port 214 may be angled with respect to the top 205. The handle 206 may have one or more inlets and one outlet.

The guide wire lumen port 212 may have an inlet 207, an outlet 209, and a bore 211 in between the inlet and the outlet 209. The inlet 207 may taper down in transitioning to the bore 211. The wide end of the taper may allow a guide wire to be inserted through the inlet 207 with ease. The outlet 209 may open up to a hollow interior 213 of a handle body 215, which may comprise a portion of the fluid conduit 103. Inner wall or walls 217 of the interior 213 may taper down in transitioning to the bore 211. The bore 211 may receive the guide wire lumen 208. The guide wire lumen 208 may pass through interior 213 and exit through the connector outlet 216. The elongate shaft 104 may go over the guide wire lumen 208. The elongate shaft 104 may be directly inserted into the connector outlet 216 or vice versa. The guide wire lumen 208 may pass through a middle of the elongate shaft 104.

The balloon inflation port 214 may have an inlet 219, an outlet 221, and a bore 223 in between the inlet 219 and the outlet 221. The inlet 219 may taper down in transitioning to the bore 223. The taper may allow a portion of the fluid conduit 103 (e.g., tubing or a connector) to be inserted into the inlet 219 and retained.

The outlet 221 may open up to the hollow interior 213 of the handle body 215. The balloon inflation port 214 may carry fluid into the interior 213 and to the elongate shaft 204. As such, the fluid conduit 103 may extend through the handle 206. The hydraulic shock arrestor 218 may be positioned on the fluid conduit 103 and within the handle 206, with a Y-connector 227 coupling the hydraulic shock arrestor 218 to the fluid conduit 103.

The inner wall 217 may be interrupted to form an inlet 222 of the hydraulic shock arrestor 218.

The hydraulic shock arrestor 218 may have a piston 256 slidably engaged with inner wall or walls 258 of the chamber 220 of the hydraulic shock arrestor 218. A portion of the inner wall 258 and a portion of the inner wall 217 may be opposite sides of the same wall. The piston 256 may create a seal with the inner walls 258.

The hydraulic shock arrestor 218 may otherwise include similar components and operate in a similar manner as the hydraulic shock arrestor 118 shown in FIGS. 1-3. For example, the hydraulic shock arrestor may include a chamber 220 having a closed end 266, a piston 256, at least one groove 260 on the outer surface 262 of the piston 256, and a seal ring 264. The hydraulic shock arrestor may include a piston retainer 255 in examples to retain the piston 256 in the chamber 220. A spring 268 may be provided in examples to absorb a pressure shock. The chamber 220 may be filled with a gas in examples, similar to the hydraulic shock arrestor 118 shown in FIGS. 1-3.

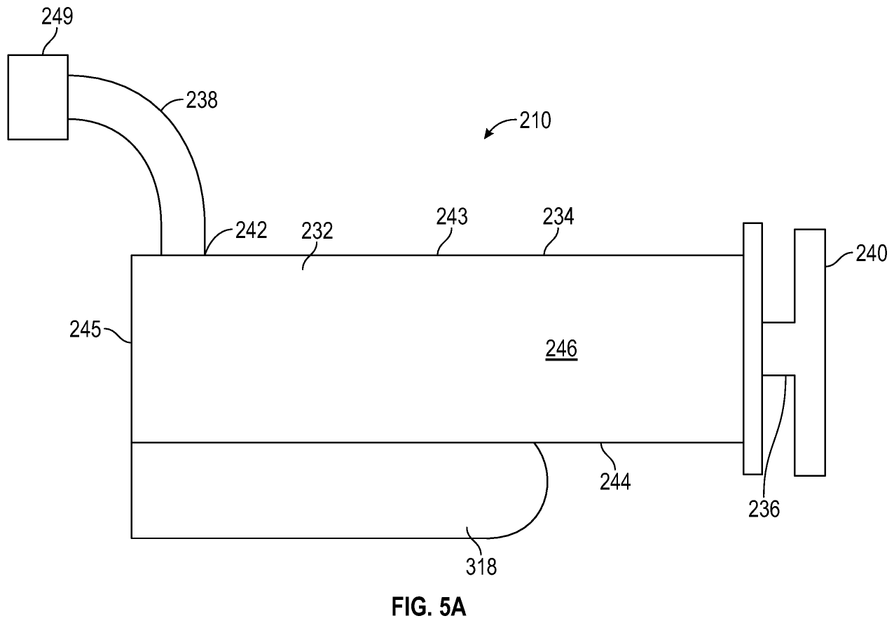

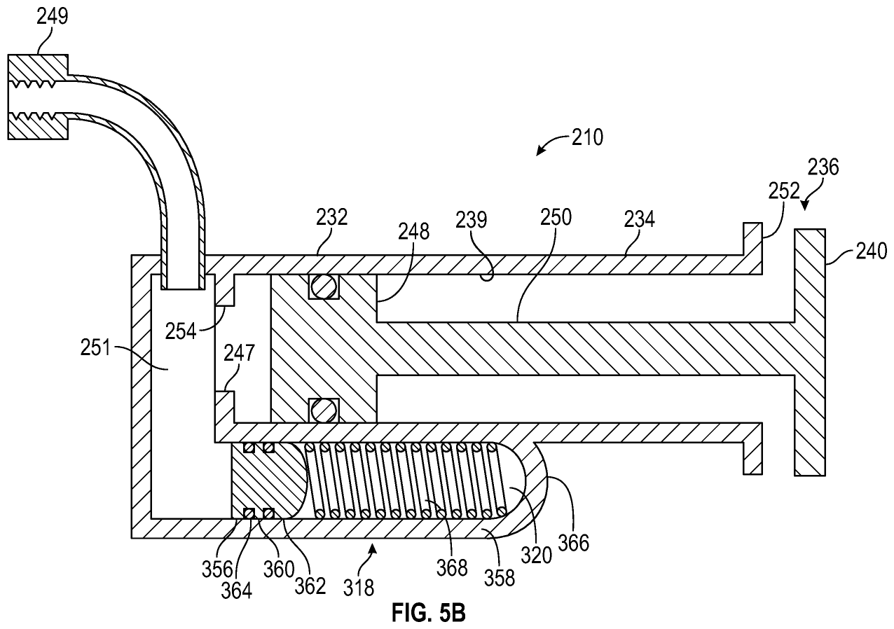

FIGS. 5A and 5B illustrate an example in which the hydraulic shock arrestor 318 is positioned on a fluid actuator 210. In the example of FIGS. 5A and 5B, the fluid actuator 210 may be a syringe as shown, including a barrel 234 and a plunger 236 slidably engaged with the barrel 234. The fluid may be expelled by lowering the plunger 236 through the barrel 234. Other examples of the fluid actuator 210 including a hydraulic shock arrestor 318 thereon may comprise an injector or a pump among other forms of fluid actuators.

In examples, the fluid actuator 210 may have the capabilities of the fluid actuator 110 shown in FIGS. 1-3, yet may have the hydraulic shock arrestor 318 positioned thereon.

The fluid actuator 210 may include a housing 232, the barrel 234, and the plunger 236. A handle 240 or pressing surface of the plunger 236 may extend out of the barrel 234. The handle 240 may be grabbed to push and pull the plunger 236.

A tube 238 may be connected to an opening 242 of the housing 232. The fluid tube 238 may extend from a top 243 of the housing 232 as shown in FIG. 5A. In some examples, the opening 242 may be on a bottom 244 of the housing 232, and the fluid tube 238 may extend from the bottom 244. The opening 242 may be proximate to a proximal end 245 of the housing 232. In some examples, the opening 242 may be at the proximal end 245, and the fluid tube 238 may extend from the proximal end 245. In some examples, the opening 242 may be on a left or right side 246 of the housing 232, and the fluid tube 238 may extend from the left or right side 246.

The fluid may exit out of the fluid tube 238 through a fluid tube outlet 249. The fluid tube outlet 249 may be connected to another component of the system such as a connector 106 (see FIG. 1) or another component such as other connectors, or the elongate shaft 104.

The hydraulic shock arrestor 318 may have an enclosing that is uniformly constructed with the housing 232. The hydraulic shock arrestor 318 may extend from the bottom 244 of the housing 232 as shown in FIG. 5A. In some examples, the hydraulic shock arrestor 318 may be located on the left or right side 246. In some examples, the hydraulic shock arrestor 318 may be located on the top 243 of the housing 232.

FIG. 5B illustrates a cross-section view of the fluid actuator 210 according to an aspect of the present disclosure. Referring to FIG. 5B, the barrel 234 may have an open connection with the hydraulic shock arrestor 318. The plunger 236 may direct incoming fluid from the barrel 234 to the integrated hydraulic shock arrestor 318 and mitigate pressure surge from a proximal end 247 of the barrel 234 when the plunger 236 is bottomed out or the plunger 236 otherwise ceases movement.

The plunger 236 may have a base 248, a shaft 250, and the handle 240. The handle 240 may be connected to the base 248 via the shaft 250. The handle 240 may extend out of the barrel 234 through a proximal end 252 of the fluid actuator 210. The base 248 may form a seal with the inner walls 239 of the barrel 234. The seal may prevent the fluid from escaping towards the proximal end 252. The sealing material may be rubber, polytetrafluoroethylene, polyethylene, or another material. The seal may be liquid proof and/or gas proof.

The barrel outlet 254 may connect the barrel 234 to a hub 251. The hub 251 may be positioned at a distal end of the barrel 234 that is connected to an open end of the hydraulic shock arrestor 318. The hub 251 may have an open connection with the hydraulic shock arrestor 318. The fluid may travel to the hydraulic shock arrestor 318 from the hub 251. An open end of the hydraulic shock arrestor 318 may be directly connected to the barrel 234 of the fluid actuator 210.

The hydraulic shock arrestor 318 may have a piston 356 slidably engaged with the inner wall or walls 358 of the chamber 320 of the hydraulic shock arrestor 318. The piston 356 may create a seal with the inner walls 358. A portion of the inner wall 358 and a portion of the inner wall 239 of the barrel 234 may be opposite sides of the same wall. The chamber 320 and the barrel 234 may accordingly share a wall.

The hydraulic shock arrestor 318 may otherwise include similar components and operate in a similar manner as the hydraulic shock arrestor 118 shown in FIGS. 1-3. For example, the hydraulic shock arrestor may include a chamber 320 having a closed end 366, a piston 356, at least one groove 360 on the outer surface 362 of the piston 356, and a seal ring 364. A spring 368 may be provided in examples to absorb a pressure shock. The chamber 320 may be filled with a gas in examples, similar to the hydraulic shock arrestor 118 shown in FIGS. 1-3.

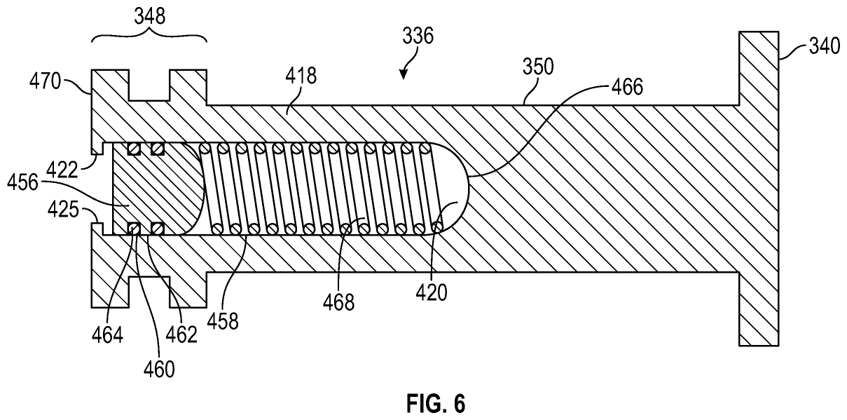

FIG. 6 illustrates an example in which the hydraulic shock arrestor 418 is positioned on the fluid actuator, yet integrated with the plunger 336. FIG. 6 illustrates an isolated cross-section view of a plunger 336 according to an example of the present disclosure.

The plunger 336 may function like the plunger 136 of FIG. 2 and plunger 236 of FIGS. 5A-5B, except that the hydraulic shock arrestor 418 may be integrated into the plunger 336.

The plunger 336 may have a base 348, a shaft 350, and a handle 340. The handle 340 may be connected to the base 348 via the shaft 350. The hydraulic shock may be mitigated by the hydraulic shock arrestor 418. The plunger 336 may be positioned within a chamber similar to other examples of fluid actuators disclosed herein.

The hydraulic shock arrestor 418 may otherwise include similar components and operate in a similar manner as the hydraulic shock arrestor 118 shown in FIGS. 1-3. For example, the hydraulic shock arrestor may include a chamber 420 having a closed end 466, a piston 456, at least one groove 460 on the outer surface 462 of the piston 456, and a seal ring 464. A spring 468 may be provided in examples to absorb a pressure shock. The chamber 420 may be filled with a gas in examples, similar to the hydraulic shock arrestor 118 shown in FIGS. 1-3.

In some examples, the chamber 420 may have a piston retainer 425 extending into the chamber 420 from the inner walls 458. The piston retainer 425 may be located at the proximal end 470. In some examples, the chamber 420 may have a bottleneck at the inlet 422 to retain the piston 456 within the chamber 420.

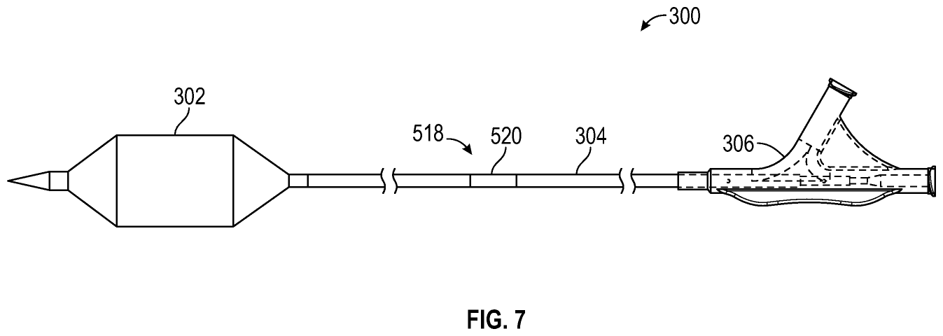

FIG. 7 illustrates a side view of a catheter system 300 having a hydraulic shock arrestor 518 including an expandable section 520 positioned on the elongate shaft 304. The catheter system 300 may have the same specifications and functionality of the catheter system 100 of FIG. 1, except the catheter system 300 may have the hydraulic shock arrestor 518 positioned on the elongate shaft 304. The catheter system 300 may further include an inflatable balloon 302 that may be configured similarly as the inflatable balloon 102 shown in FIG. 1, and a connector 306 that may be configured similarly as the connector shown in FIG. 1.

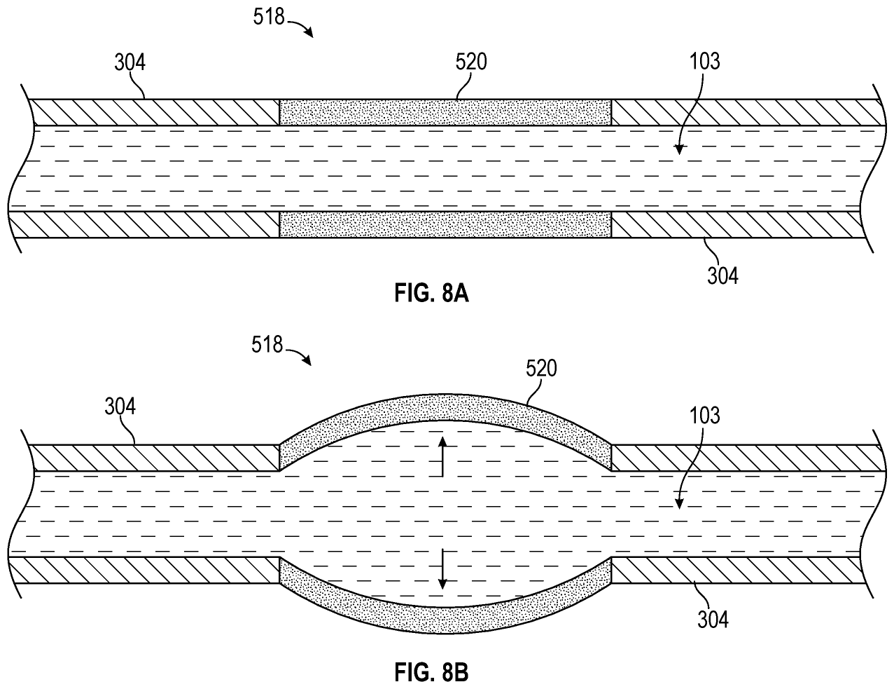

The hydraulic shock arrestor 518 may have an expandable section 520 made out of a material that is configured to deflect due to a force. FIG. 8A for example, illustrates a close up view of the hydraulic shock arrestor 518. The expandable section 520 may surround the fluid conduit 103. The expandable section 520 may be configured to receive a force of the hydraulic shock radially outward upon the expandable section 520 and deflect due to the force. FIG. 8A, for example, illustrates a configuration of the expandable section 520 without a hydraulic shock being applied to the expandable section 520. FIG. 8B, however, illustrates the hydraulic shock being applied with the expandable section 520 deflecting to mitigate the pressure surge within the fluid conduit 103.

In examples, the expandable section 520 may be made of a material that is flexible and configured to deflect. The expandable section 520 may be biased to return to a undeflected configuration as shown in FIG. 8A. For example, such materials may comprise elastomeric materials. In examples, such materials may comprise viscoelastic materials. Viscoelastic materials may behave like a liquid and a solid material and have a time-dependent strain. The viscoelastic material may be an amorphous polymer, a semi-crystalline polymer, or a biopolymer, among other types of materials. When fluid flows within the elongate shaft 304 without a pressure surge, the hydraulic shock arrestor 518 may be stiff and retain its form. When water hammer effect causes a pressure surge in the catheter system 300, the hydraulic shock arrestor 518 may expand to absorb the shock from the pressure surge. When the pressure surge is contained, the hydraulic shock arrestor 518 may return to its original form.

Referring back to FIG. 1, in some examples, the hydraulic shock arrestor may be positioned on a stopcock such as the valve switch 132 shown in FIG. 1. The hydraulic shock arrestor accordingly may be configured to mitigate pressure surge within the fluid conduit that may be caused by the valve switch 132 being closed, which may produce a hydraulic shock. The hydraulic shock arrestor on the stopcock may mitigate the hydraulic shock produced by such an operation, or may be utilized to mitigate pressure surge from other sources such as a fluid actuator.

Combinations of hydraulic shock arrestors may be utilized. For example, multiple hydraulic shock arrestors in multiple positions on the catheter system may be utilized in examples.

In examples herein, the inflation fluid may comprise an incompressible fluid. In examples, the fluid may comprise a contrast-saline mixture.

As discussed, the catheter system may be utilized for a variety of applications, including dilating a surface within a patient's body upon inflation of the inflatable balloon. In examples, an implant may be expanded that is positioned upon the inflatable balloon. The implant may be expanded within the patient's body upon inflation of the inflatable balloon.

The implant may have a variety of configurations. In examples, the implant may have a configuration as shown in FIGS. 9-11 among other forms of implants.

FIG. 9 illustrates a perspective view of a prosthetic implant 610 in the form of a replacement heart valve. The prosthetic implant 610 may be configured to be deployed within a portion of a patient's body. The prosthetic implant 610, for example, may be deployed within a native heart valve annulus, which may comprise a native aortic valve, or in examples may comprise a native mitral, tricuspid, or pulmonary valve. In examples, the implant 610 may have other forms, and may comprise a stent or other form of medical implant as desired.

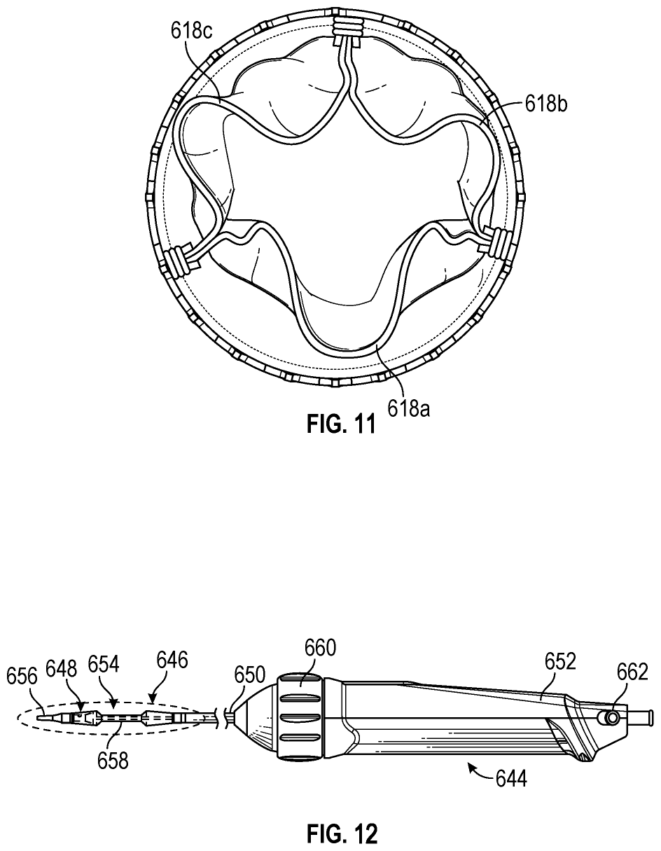

The prosthetic implant 610 may include a proximal end 612 and a distal end 614, and a length therebetween. The prosthetic implant 610 may include a body in the form of a frame 616. The prosthetic implant 610 may further include one or more of a plurality of leaflets 618a— c (marked in FIGS. 10 and 11) coupled to the frame 616 and may include a skirt 620 covering an outer surface of a distal portion of the frame 616. The leaflets 618a—c may move back and forth between open and closed positions or states or configurations to replicate the motion of a native valve.

The leaflets 618a—c may be configured to open and close during operation such that the proximal end 612 of the implant 610 forms an outflow end of the implant 610, and the distal end 614 of the implant 610 forms an inflow end of the implant 610. The leaflets 618a—c may be configured to impede fluid flow in an opposite direction from the outflow end to the inflow end of the implant 610 when the leaflets 618a—c are in a closed position.

The frame 616 may comprise a plurality of struts 622 connected at junctures 624. A plurality of openings 626 may be positioned between the struts 622. The openings 626 may be configured to reduce the overall weight of the frame 616, and also allow the frame 616 to be compressed to reduce a diameter of the frame 616 and be expanded to increase a diameter of the frame 616. The frame 616 may be configured to be radially compressed and axially lengthened while being radially compressed. The struts 622 may be configured such that as the frame 616 is compressed to reduce a diameter of the frame 616, the length of the frame 616 may increase. Also, as the frame 616 is expanded to increase the diameter of the frame 616, the length of the frame 616 may decrease. The frame 616 may be compressed in a variety of manners, including use of a crimping device, and may be expanded in a variety of manners, including being expanded with an inflatable balloon as disclosed herein.

The configuration of the implant shown in FIGS. 9-11 may be varied in examples.

The implant 610 may be configured to be delivered to an implantation site utilizing a catheter system. FIG. 12, for example, illustrates an example of a catheter system in the form of a delivery apparatus 644 that may be utilized to deliver the implant 610 to a desired implantation site. The delivery apparatus 644 may include an elongate shaft 646 having a distal end portion 648 and a proximal end portion 650. The distal end portion may be configured to couple to the inflatable balloon 658. The proximal end portion 650 may couple to a connector in the form of a handle 652. The distal end portion 648 may include an implant retention area 654 and a distal tip that may include a nose cone 656. The distal end portion 648 may further couple to the inflatable balloon 658 (shown in a deflated state in FIG. 12). The inflatable balloon 658 may be for insertion within a patient's body and configured to be inflated with a fluid within the patient's body.

The delivery apparatus 644 may be configured to be positioned within a crimping device to crimp the implant 610 to the implant retention area 654. The elongate shaft 646 may be positioned within the crimping device. The inflatable balloon 658 may be configured for the implant 610 to be crimped upon.

The handle 652 may be configured for a user to grip to operate the delivery apparatus 644 and to maneuver the delivery apparatus 644 through the vasculature of the patient's body. For example, the handle 652 may be moved distally to advance the elongate shaft 646 distally within the patient's body and may be moved proximally to retract the elongate shaft 646 proximally within the patient's body. As such, the implant retention area 654 and accordingly the implant 610 may be moved and positioned with the operation of the handle 652.

A control mechanism 660 may further be coupled to the handle 652. The control mechanism 660 may be configured to be operated to bend the elongate shaft 646 as desired. For example, one or more pull tethers may extend along the elongate shaft 646 and operation of the control mechanism 660 may push or pull the one or more pull tethers to cause the elongate shaft 646 to bend. The bending of the elongate shaft 646 accordingly may be controlled by the control mechanism 660. As shown in FIG. 12, the control mechanism 660 may comprise a rotatable body in the form of a control knob that may be rotated to push or pull the pull tether and cause the elongate shaft 646 to bend. Other forms of control mechanisms may be utilized as desired.

The catheter system including the delivery apparatus 644 may be configured similarly as examples of catheter systems disclosed herein. For example, the inflatable balloon 658 may be configured similarly as inflatable balloons disclosed herein, the elongate shaft 646 may be configured similarly as elongate shafts disclosed herein, and the handle 652 may be configured similarly as handles disclosed herein.

The catheter system may include a fluid conduit as disclosed herein. The catheter system may include a hydraulic shock arrestor as disclosed herein, which may have a configuration and position of any examples of hydraulic shock arrestor disclosed herein. For example, the hydraulic shock arrestor may comprise a separate component, may be positioned on the handle 652, may be positioned on the elongate shaft 646, or may be positioned on a fluid actuator that may be utilized with the catheter system. The hydraulic shock arrestor may be positioned along the fluid conduit. A fluid actuator may be utilized to inflate the inflatable balloon 658.

In examples, a fluid port 662 may be coupled to the handle 652 and may be utilized to transfer fluid to and from the balloon 658 as desired. The fluid port 662 may comprise a component of the fluid conduit. The configuration of the handle 652 may be varied in other examples as desired.

FIGS. 13 and 14 illustrate an exemplary operation of deploying the prosthetic implant 610 in the form of a prosthetic heart valve. The prosthetic implant 610 for example, may be positioned at the inflation site, which may be an aortic valve 700 as shown herein, or another location as desired. FIG. 14 illustrates the inflatable balloon 658 being inflated utilizing a fluid actuator. The prosthetic implant 610 is expanded upon the inflatable balloon 658 and deployed to the implantation site. The prosthetic implant 610 may either be positioned upon the inflatable balloon 658 upon insertion into the patient's body, or may be slid onto the inflatable balloon 658 following insertion into the patient's body. Various other delivery methods may be utilized as desired.

The inflation may be rapid, which may result in a possible hydraulic shock due to a rapid cessation of the flow of the fluid upon inflation. In the event of a hydraulic shock, a hydraulic shock arrestor as disclosed herein may be utilized to mitigate a pressure surge within the fluid conduit, as discussed herein. The inflatable balloon 658 may then be deflated and withdrawn from the patient's body with the prosthetic implant 610 remaining in position.

Various other methods may be utilized according to examples herein. For example, dilation of a portion of the vasculature (including native leaflets of a heart valve) may occur, among other methods.

The features of the examples disclosed herein may be implemented independently or in combination with other features disclosed herein.

As discussed, various forms of implants may be utilized with the examples disclosed herein, including prosthetic heart valves, or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The delivery apparatuses and the systems disclosed herein may be used in transcatheter aortic valve implantation (TAVI) or replacement of other native heart valves (e.g., mitral, tricuspid, or pulmonary). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

Features of examples may be modified, substituted, excluded, or combined across examples as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

The features of the examples disclosed herein may be implemented independently, or independent of other components disclosed herein. The various apparatuses of the system may be implemented independently.
Example 1: A catheter system. The catheter system may include an inflatable balloon for insertion within a patient's body and configured to be inflated with a fluid within the patient's body; an elongate shaft configured to extend within the patient's body and having a distal end portion configured to couple to the inflatable balloon and a proximal end portion; a fluid conduit configured to extend between the inflatable balloon and a fluid actuator, and configured to convey movement of the fluid to the inflatable balloon from the fluid actuator to inflate the inflatable balloon; and a hydraulic shock arrestor configured to mitigate pressure surge within the fluid conduit.
Example 2: The catheter system of any example herein, in particular Example 1, further comprising a connector configured to couple the hydraulic shock arrestor to the elongate shaft.
Example 3: The catheter system of any example herein, in particular Example 2, wherein the connector is a releasable connector.
Example 4: The catheter system of any example herein, in particular Example 2 or Example 3, wherein the connector is a luer connector.
Example 5: The catheter system of any example herein, in particular Examples 1-4, wherein the fluid conduit extends along the elongate shaft, and the hydraulic shock arrestor is configured to be positioned on the fluid conduit at a position between the elongate shaft and the fluid actuator.
Example 6: The catheter system of any example herein, in particular Examples 1-5, wherein at least a portion of the fluid conduit extends through the elongate shaft.
Example 7: The catheter system of any example herein, in particular Examples 1-6, wherein the elongate shaft includes a guide wire lumen extending along the elongate shaft and configured to receive a guide wire.
Example 8: The catheter system of any example herein, in particular Examples 1-7, wherein the hydraulic shock arrestor is coupled to a tube having an inlet connector with a first opening and an outlet connector with a second opening, the outlet connector configured to couple to the elongate shaft and the inlet connector configured to couple to the fluid actuator, and the fluid conduit extending from the first opening to the second opening.
Example 9: The catheter system of any example herein, in particular Examples 1-8, further comprising a handle coupled to the proximal end portion of the elongate shaft, the hydraulic shock arrestor being positioned on the handle.
Example 10: The catheter system of any example herein, in particular Example 9, wherein the fluid conduit extends through the handle.
Example 11: The catheter system of any example herein, in particular Example 10, wherein a Y-connector couples the hydraulic shock arrestor to the fluid conduit.
Example 12: The catheter system of any example herein, in particular Examples 1-11, further comprising the fluid actuator.
Example 13: The catheter system of any example herein, in particular Example 12, wherein the fluid actuator is positioned at a proximal end portion of the fluid conduit.
Example 14: The catheter system of any example herein, in particular Example 12 or Example 13, further comprising a tube extending from the fluid actuator to the hydraulic shock arrestor, the tube surrounding the fluid conduit.
Example 15: The catheter system of any example herein, in particular Examples 12-14, wherein the hydraulic shock arrestor is positioned on the fluid actuator.
Example 16: The catheter system of any example herein, in particular Examples 12-15, wherein the fluid actuator includes a plunger slidably engaged with a barrel of the fluid actuator, the fluid being expelled from the barrel by lowering the plunger through the barrel.
Example 17: The catheter system of any example herein, in particular Example 16, wherein the hydraulic shock arrestor is positioned on the fluid actuator such that an open end of the hydraulic shock arrestor is directly connected to the barrel of the fluid actuator.
Example 18: The catheter system of any example herein, in particular Example 16 or Example 17, further comprising a hub positioned at a distal end of the barrel that is connected to an open end of the hydraulic shock arrestor.
Example 19: The catheter system of any example herein, in particular Examples 16-18, further comprising a hub positioned at a distal end of the barrel that is connected to an open end of the hydraulic shock arrestor.
Example 20: The catheter system of any example herein, in particular Examples 16-19, wherein the hydraulic shock arrestor is integrated with the plunger.
Example 21: The catheter system of any example herein, in particular Examples 1-20, wherein the hydraulic shock arrestor includes a chamber and a piston located within the chamber, the piston being slidably and sealably engageable with inner walls of the chamber.
Example 22: The catheter system of any example herein, in particular Example 21, wherein the chamber is filled with gas to resist pressure.
Example 23: The catheter system of any example herein, in particular Example 22, wherein the piston includes a first side configured to be in contact with the gas and a second side configured to be in contact with the fluid within the fluid conduit.
Example 24: The catheter system of any example herein, in particular Examples 21-23, wherein the piston is sealably engageable with the inner walls with at least one seal ring configured to create a seal between the piston and the inner walls.
Example 25: The catheter system of any example herein, in particular Examples 21-24, wherein the hydraulic shock arrestor includes an inner spring located between the piston and the chamber, the inner spring configured to absorb pressure.
Example 26: The catheter system of any example herein, in particular Examples 1-25, wherein the hydraulic shock arrestor includes an expandable section positioned on the elongate shaft.
Example 27: The catheter system of any example herein, in particular Examples 1-26, wherein the hydraulic shock arrestor is positioned on a stopcock.
Example 28: The catheter system of any example herein, in particular Examples 1-27, wherein the hydraulic shock arrestor is a water hammer arrestor.
Example 29: The catheter system of any example herein, in particular Examples 1-28, wherein the fluid is an incompressible fluid.
Example 30: The catheter system of any example herein, in particular Example 29, wherein the incompressible fluid is a contrast-saline mixture.
Example 31: A method comprising: extending an elongate shaft of a catheter system within a portion of a patient's body, the catheter system including: an inflatable balloon coupled to a distal end portion of the elongate shaft and configured to be inflated with a fluid, a fluid actuator configured to move the fluid to inflate the inflatable balloon, a fluid conduit extending between the inflatable balloon and the fluid actuator and configured to convey movement of the fluid to the inflatable balloon from the fluid actuator, and a hydraulic shock arrestor configured to mitigate pressure surge within the fluid conduit. The method may comprise positioning the inflatable balloon at an inflation site within the patient's body. The method may comprise inflating the inflatable balloon utilizing the fluid actuator.
Example 32: The method of any example herein, in particular Example 31, wherein a connector couples the hydraulic shock arrestor to the elongate shaft.
Example 33: The method of any example herein, in particular Example 32, wherein the connector is a releasable connector.
Example 34: The method of any example herein, in particular Example 32 or Example 33, wherein the connector is a luer connector.
Example 35: The method of any example herein, in particular Examples 31-34, wherein the fluid conduit extends along the elongate shaft, and the hydraulic shock arrestor is positioned on the fluid conduit at a position between the elongate shaft and the fluid actuator.
Example 36: The method of any example herein, in particular Examples 31-35, wherein at least a portion of the fluid conduit extends through the elongate shaft.
Example 37: The method of any example herein, in particular Examples 31-36, wherein the elongate shaft includes a guide wire lumen extending along the elongate shaft.
Example 38: The method of any example herein, in particular Examples 31-37, wherein the hydraulic shock arrestor is coupled to a tube having an inlet connector with a first opening and an outlet connector with a second opening, and the outlet connector is coupled to the elongate shaft, and the inlet connector is coupled to the fluid actuator, and the fluid conduit extends from the first opening to the second opening.
Example 39: The method of any example herein, in particular Examples 31-38, wherein a handle is coupled to a proximal end portion of the elongate shaft and the hydraulic shock arrestor is positioned on the handle.
Example 40: The method of any example herein, in particular Example 39, wherein the fluid conduit extends through the handle.
Example 41: The method of any example herein, in particular Examples 31-40, wherein the fluid actuator is positioned at a proximal end portion of the fluid conduit.
Example 42: The method of any example herein, in particular Examples 31-41, wherein a tube extends from the fluid actuator to the hydraulic shock arrestor and the tube surrounds the fluid conduit.
Example 43: The method of any example herein, in particular Examples 31-42, wherein the hydraulic shock arrestor is positioned on the fluid actuator.
Example 44: The method of any example herein, in particular Examples 31-42, wherein the fluid actuator includes a plunger slidably engaged with a barrel of the fluid actuator, and the method further comprises expelling the fluid from the barrel by lowering the plunger through the barrel.
Example 45: The method of any example herein, in particular Example 44, wherein the hydraulic shock arrestor is positioned on the fluid actuator such that an open end of the hydraulic shock arrestor is directly connected to the barrel of the fluid actuator.
Example 46: The method of any example herein, in particular Example 44 or Example 45, wherein a hub is positioned at a distal end of the barrel that is connected to an open end of the hydraulic shock arrestor.
Example 47: The method of any example herein, in particular Examples 44-46, wherein the hydraulic shock arrestor has a chamber, and the chamber and the barrel share a wall.
Example 48: The method of any example herein, in particular Examples 44-47, wherein the hydraulic shock arrestor is integrated with the plunger.
Example 49: The method of any example herein, in particular Examples 31-48, wherein the hydraulic shock arrestor includes a chamber and a piston located within the chamber, the piston being slidably and sealably engageable with inner walls of the chamber.
Example 50: The method of any example herein, in particular Example 49, wherein the chamber is filled with gas to resist pressure.
Example 51: The method of any example herein, in particular Example 50, wherein the piston includes a first side configured to be in contact with the gas and a second side configured to be in contact with the fluid within the fluid conduit.
Example 52: The method of any example herein, in particular Examples 49-51, wherein the piston is sealably engageable to the inner walls with at least one seal ring configured to create a seal between the piston and the inner walls.
Example 53: The method of any example herein, in particular Examples 49-52, wherein the hydraulic shock arrestor includes an inner spring located between the piston and the chamber, the inner spring configured to absorb pressure.
Example 54: The method of any example herein, in particular Examples 31-53, wherein the hydraulic shock arrestor includes an expandable section positioned on the elongate shaft.
Example 55: The method of any example herein, in particular Examples 31-54, wherein the hydraulic shock arrestor is positioned on a stopcock.
Example 56: The method of any example herein, in particular Examples 31-55, wherein the hydraulic shock arrestor is a water hammer arrestor.
Example 57: The method of any example herein, in particular Examples 31-56, wherein the fluid is an incompressible fluid.
Example 58: The method of any example herein, in particular Examples 31-57, further comprising dilating a surface within the patient's body upon inflation of the inflatable balloon.
Example 59: The method of any example herein, in particular Examples 31-58, further comprising expanding an implant positioned upon the inflatable balloon within the patient's body upon inflation of the inflatable balloon.
Example 60: The method of any example herein, in particular Example 59, wherein the implant comprises a prosthetic heart valve.

Any of the features of any of the examples, including but not limited to any of the first through sixtieth examples referred to above, is applicable to all other aspects and embodiments identified herein, including but not limited to any embodiments of any of the first through sixtieth examples referred to above. Moreover, any of the features of an embodiment of the various examples, including but not limited to any embodiments of any of the first through sixtieth aspects referred to above, is independently combinable, partly or wholly with other examples described herein in any way, e.g., one, two, or three or more examples may be combinable in whole or in part. Further, any of the features of the various examples, including but not limited to any embodiments of any of the first through sixtieth examples referred to above, may be made optional to other examples. Any example of a method can be performed by a system or apparatus of another example, and any aspect or embodiment of a system or apparatus can be configured to perform a method of another aspect or embodiment, including but not limited to any embodiments of any of the first through sixtieth examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A catheter delivery system for delivering a prosthetic heart valve to a native heart valve, the catheter delivery system comprising:
an inflatable balloon for insertion within a patient's body and configured to be inflated with a fluid within the patient's body, the inflatable balloon including an outer surface having a retention area for retaining the prosthetic heart valve;
an elongate catheter shaft configured to extend within the patient's body and having a distal end portion configured to couple to the inflatable balloon and a proximal end portion;
a fluid conduit configured to extend between the inflatable balloon and a fluid actuator, and configured to convey movement of the fluid to the inflatable balloon from the fluid actuator to inflate the inflatable balloon to thereby expand the prosthetic heart valve; and
a hydraulic shock arrestor configured to mitigate pressure surge within the fluid conduit.

2. The catheter delivery system of claim 1, wherein the fluid conduit extends along the elongate catheter shaft, and the hydraulic shock arrestor is configured to be positioned on the fluid conduit at a position between the elongate catheter shaft and the fluid actuator.

3. The catheter delivery system of claim 1, wherein the elongate catheter shaft includes a guide wire lumen extending along the elongate catheter shaft and configured to receive a guide wire.

4. The catheter delivery system of claim 1, wherein the hydraulic shock arrestor is coupled to a tube having an inlet connector with a first opening and an outlet connector with a second opening, the outlet connector configured to couple to the elongate catheter shaft and the inlet connector configured to couple to the fluid actuator, and the fluid conduit extending from the first opening to the second opening.

5. The catheter delivery system of claim 1, further comprising a handle coupled to the proximal end portion of the elongate catheter shaft, the hydraulic shock arrestor being positioned on the handle.

6. The catheter delivery system of claim 1, further comprising the fluid actuator.

7. The catheter delivery system of claim 6, wherein the fluid actuator is positioned at a proximal end portion of the fluid conduit.

8. The catheter delivery system of claim 6, further comprising a tube extending from the fluid actuator to the hydraulic shock arrestor, the tube surrounding the fluid conduit.

9. The catheter delivery system of claim 6, wherein the hydraulic shock arrestor is positioned on the fluid actuator.

10. The catheter delivery system of claim 6, wherein the fluid actuator includes a plunger slidably engaged with a barrel of the fluid actuator, the fluid being expelled from the barrel by lowering the plunger through the barrel.

11. The catheter delivery system of claim 10, wherein the hydraulic shock arrestor is positioned on the fluid actuator such that an open end of the hydraulic shock arrestor is directly connected to the barrel of the fluid actuator.

12. The catheter delivery system of claim 10, further comprising a hub positioned at a distal end of the barrel that is connected to an open end of the hydraulic shock arrestor.

13. The catheter delivery system of claim 10, wherein the hydraulic shock arrestor has a chamber, and the chamber and the barrel share a wall.

14. The catheter delivery system of claim 10, wherein the hydraulic shock arrestor is integrated with the plunger.

15. The catheter delivery system of claim 1, further comprising the prosthetic heart valve, wherein the prosthetic heart valve includes a frame and a plurality of leaflets coupled to the frame.

16. A method of delivering a prosthetic heart valve to a native heart valve, the method comprising:
extending an elongate catheter shaft of a catheter delivery system within a portion of a patient's body, the catheter delivery system including:
an inflatable balloon coupled to a distal end portion of the elongate catheter shaft and configured to be inflated with a fluid, the inflatable balloon including an outer surface having a retention area for retaining the prosthetic heart valve,
a fluid actuator configured to move the fluid to inflate the inflatable balloon,
a fluid conduit extending between the inflatable balloon and the fluid actuator and configured to convey movement of the fluid to the inflatable balloon from the fluid actuator, and
a hydraulic shock arrestor configured to mitigate pressure surge within the fluid conduit;
positioning the inflatable balloon at the native heart valve within the patient's body; and
inflating the inflatable balloon utilizing the fluid actuator with the prosthetic heart valve positioned on the outer surface of the inflatable balloon to thereby expand the prosthetic heart valve at the native heart valve.

17. The method of claim 16, wherein the hydraulic shock arrestor is a water hammer arrestor.

18. The method of claim 16, wherein the hydraulic shock arrestor is configured to mitigate pressure surge caused by rapid cessation of flow of the fluid.

19. The method of claim 16, wherein the fluid actuator includes a plunger slidably engaged within a barrel of the fluid actuator, and the hydraulic shock arrestor is configured to mitigate pressure surge caused by the plunger bottoming out within the barrel.

20. The method of claim 16, wherein the native heart valve is an aortic heart valve.
